SEQUENCE LISTING (7 sequences)

SEQ ID NO: 1 - protein, 108 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLELKR

SEQ ID NO: 2 - protein, 130 aa, synthetic construct
EVQLQQSGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTTVTVSSYVTVSSQDPA

SEQ ID NO: 3 - protein, 68 aa, synthetic construct
FWVLVVVGGVLACYSLLVTVAFIIFWVRSKRSRLLHSDYMNMTPRRPGPTRKHYQPYAPPRDFAAYRS

SEQ ID NO: 4 - protein, 114 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGP

SEQ ID NO: 5 - protein, 236 aa, synthetic construct
EPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPK

SEQ ID NO: 6 - protein, 162 aa, synthetic construct
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 7 - protein, 404 aa, synthetic construct
MLEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSGVDGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSASRA

(19) United States
(12) Patent Application Publication
(10) Pub. No.: US 2025/0186587 A1
(43) Pub. Date: Jun. 12, 2025

(54) CD19 CAR NK CELLS FOR USE IN METHODS OF TREATING CANCER

(71) Applicant: TAKEDA PHARMACEUTICAL COMPANY LIMITED, Osaka (JP)

(72) Inventors: Leopold Sellner, Lexington, MA (US); Siddha Narayan Kasar, Lexington, MA (US); Mary E. Carsillo, Lexington, MA (US); Michael David Curley, Lexington, MA (US)

(21) Appl. No.: 18/954,741

(22) Filed: Nov. 21, 2024

Related U.S. Application Data
(63) Continuation of application No. PCT/IB2023/055311, filed on May 23, 2023.
(60) Provisional application No. 63/348,220, filed on Jun. 2, 2022.
(60) Provisional application No. 63/344,962, filed on May 23, 2022.

(51) Int. Cl. A61K 40/15; A61K 40/42; A61K 45/06; A61P 35/00; C12N 5/0783

(52) U.S. Cl. CPC A61K 40/15; A61K 40/4211; A61K 45/06; A61P 35/00; C12N 5/0646

(57) ABSTRACT

The present disclosure provides, among other things methods and compositions for the treatment of cancer comprising administering CD19 CAR cord blood derived natural killer (CB-NK) cells to a patient in need thereof.

### CROSS-REFERENCE TO RELATED APPLICATIONS

This application is a Continuation Application of International Application No. PCT/IB2023/055311, filed on May 23, 2023, which claims priority to, and the benefit of, U.S. Provisional Application No. 63/348,220, filed on Jun. 2, 2022 and 63/344,962, filed on May 23, 2022, the contents of each of which are hereby incorporated by reference in their entireties.

### INCORPORATION BY REFERENCE OF SEQUENCE LISTING

The instant application is being filed with an electronically filed Sequence Listing in XML format. The sequence listing file entitled MIL-020US1_SL.xml, was created on Jan. 29, 2025, and is 8 kilobytes in size; the information in electronic format of the Sequence Listing is incorporated herein by reference in its entirety.

### BACKGROUND

Chimeric antigen receptor (CAR) T cell therapy has been shown to be effective in the treatment of certain cancers. Clinical trials have shown that 40-90% complete remission (CR) can be achieved in pediatric and adult patients treated with CD19-directed CAR T-cells. However, 30-60% of patients relapse after CD19 CAR T-cell treatment, and among those, 20-90% are CD19-negative relapse. The eligible treatment options for post-CAR relapse are limited, making it more difficult to achieve CR and improve survival rate. There is an unmet medical need for patients following relapse after exposure to a previous CD19-targeted therapy.

### SUMMARY

The present application is based, at least in part, on the discovery that CD19-directed genetically modified NK cell immunotherapy (e.g., CD19 CAR+NK cells, CD19 CAR+ viable NK cells and/or allogenic cord blood derived CD19 CAR NK+ cells) described herein has efficacy in patients with a history of anti-CD19 therapy (i) in relapsed cancer that is CD19-positive, and (ii) relapsed cancer that is CD19-negative. Without wishing to be bound by theory, NK cells may be able to kill tumor cells upon target antigen downregulation via innate immune receptor-mediated killing.

In one aspect, the present invention provides a method of treating cancer in an individual, comprising the step of administering a therapeutically effective amount of CD19-CAR+ viable cord blood natural killer (CB-NK) cells to the individual, wherein the individual has a history of anti-CD19 therapy.

In one aspect, the present invention provides a method of treating cancer in an individual, comprising the step of administering a therapeutically effective amount of CD19-CAR+ viable cord blood natural killer (CB-NK) cells to the individual, wherein the individual has previously received an anti-CD19 therapy.

In some embodiments, the individual has failed 2 or more prior lines of systemic therapy prior to administration of CD19-CAR+ viable CB-NK cells described herein.

In some embodiments, the therapeutically effective amount of immune cells will depend on the individual being treated, the severity and type of the cancer being treated.

In some embodiments, the CD19-CAR+ viable CB-NK cells are administered at a dose of between 200×10⁶ to 800×10⁶ CD19-CAR+ viable CB-NK cells. In some embodiments, CD19-CAR+ viable CB-NK cells are administered at a dose of at least 200×10⁶, at least 300×10⁶, at least 400×10⁶, at least 500×10⁶, at least 600×10⁶, at least 700×10⁶, or at least 800×10⁶ CD19-CAR+ viable CB-NK cells.

In some embodiments, the CD19-CAR+ viable CB-NK cells are administered at a dose of 800×10⁶ CD19-CAR+ viable CB-NK cells. In some embodiments, the CD19-CAR+ viable CB-NK cells are administered at a dose of 700×10⁶ CD19-CAR+ viable CB-NK cells. In some embodiments, the CD19-CAR+ viable CB-NK cells are administered at a dose of 600×10⁶ CD19-CAR+ viable CB-NK cells. In some embodiments, the CD19-CAR+ viable CB-NK cells are administered at a dose of 500×10⁶ CD19-CAR+ viable CB-NK cells. In some embodiments, the CD19-CAR+ viable CB-NK cells are administered at a dose of 400×10⁶ CD19-CAR+ viable CB-NK cells. In some embodiments, the CD19-CAR+ viable CB-NK cells are administered at a dose of 300×10⁶ CD19-CAR+ viable CB-NK cells. In some embodiments, the CD19-CAR+ viable CB-NK cells are administered at a dose of 200×10⁶ CD19-CAR+ viable CB-NK cells.

In some embodiments, the anti-CD19 therapy (also referred to as CD19 targeted therapy herein) is therapy in which CD19-targeted chimeric antigen receptor (CAR) T cells, CD19-targeted antibody-drug-conjugates and/or CD19-targeted antibodies are administered to an individual. In one embodiment, the anti-CD19 therapy is CD19-targeted chimeric antigen receptor (CAR) T therapy.

In some embodiments, the CD19-CAR+ viable CB-NK cells are derived from cord blood.

In some embodiments, the individual is administered lymphodepleting chemotherapy intravenously followed by administration of CD19-CAR+ viable CB-NK cells.

In some embodiments, the individual previously received anti-CD19 CAR-T therapy more than three months prior to administration of the CD19-CAR+ viable CB-NK cells.

In some embodiments, the cancer is a solid tumor or is not a solid tumor.

In some embodiments, the cancer is of the lung, brain, breast, blood, skin, pancreas, liver, colon, head and neck, kidney, thyroid, stomach, spleen, gallbladder, bone, ovary, testes, endometrium, prostate, rectum, anus, cervix, or is hematological.

In some embodiments, the cancer is relapsed or refractory B-cell Non-Hodgkin Lymphoma, including large B-cell lymphoma and indolent non-Hodgkin lymphoma. In some embodiments, the cancer is chronic lymphocytic leukemia (CLL). In some embodiments, the cancer is acute lymphoblastic leukemia (ALL). In some embodiments, the cancer is not limited to CD19 and CD20 double positive cancer.

In some embodiments, the individual is a human.

In some embodiments, the individual is administered one or more additional cancer therapies.

In some embodiments, the additional cancer therapy is surgery, radiation, chemotherapy, hormone therapy, immunotherapy, or a combination thereof.

In some embodiments, the method comprises a step of diagnosing cancer in the individual.

In some embodiments, the method comprises a step of generating the CD19-CAR+ viable CB-NK cells.

In some embodiments, the CD19-CAR+ viable CB-NK cells are autologous with respect to the individual.

In some embodiments, the CD19-CAR+ viable CB-NK cells are allogeneic with respect to the individual.

In some embodiments, the CD19-CAR+ viable CB-NK cells administered to the individual intracranially, by injection, intravenously, intraarterially, intraperitoneally, intratracheally, intratumorally, intramuscularly, endoscopically, intralesionally, intracranially, percutaneously, subcutaneously, regionally, by perfusion, in a tumor microenvironment, or a combination thereof.

In some embodiments, the CD19-CAR+ viable (NK) cells comprise one or more exogenously provided interleukins (IL).

In some embodiments, the IL is selected from the group consisting of IL-12, IL-15, IL-21, IL-2, IL-18, IL-7, the p35 and p40 subunits of IL-12 artificially linked together with a linker, and a combination thereof.

In some embodiments, the IL is IL-15.

In some embodiments, the IL is secreted, tethered, or membrane bound in the cell.

In some embodiments, the exogenously provided IL is expressed from a vector in the cells and/or wherein the NK cells are cultured in the presence of one or more IL.

In some embodiments, the NK cell comprises a suicide gene.

In some embodiments, the CD19-CAR+ viable (NK) cells further comprise an iCaspase9 suicide gene.

In some embodiments, the NK cells are cord blood derived or induced pluripotent stem cell (iPSC) derived NK cells. In some embodiments, the NK cells are cord blood derived NK cells. In some embodiments, the NK cells are iPSC derived NK cells.

In some embodiments, the CD19-CAR+ viable CB-NK cells were previously frozen and thawed.

In some embodiments, the NK cells are genetically engineered cord blood NK cells.

In some embodiments, the NK cells are genetically engineered with a chimeric antigen receptor (CAR) that binds CD19. In some embodiments, the NK cells are not genetically engineered to target tumor antigens other than CD19. In some embodiments, the CD19 CAR CB-NK cells described herein are genetically engineered to target only CD19 expressing cells. In some embodiments, treatment with the CD19 CAR CB-NK described herein has reduced side effects on normal cells or cells expressing a tumor antigen other than CD19. In some embodiments, the CD19 CAR CB-NK cells do not express a CD16 (hnCD16) Fc receptor.

In some embodiments, the genetically engineered cord blood NK cells comprise human cord blood-derived NK cells (CB-NK) transduced with a retroviral vector expressing an iCaspase9, a CD19-CAR and an IL-15. In some embodiments, the genetically engineered cord blood NK cells comprise human cord blood-derived NK cells (CB-NK) transduced with non-viral vector expressing an iCaspase9, a CD19-CAR and an IL-15.

In some embodiments, the genetically engineered cord blood NK cells include a CD19-CAR comprising an anti-CD19 binding domain, a transmembrane domain such as the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154 and an intracellular signaling domain such as an intracellular signaling domain FcR gamma, FcR beta, CD3 gamma, CD3 delta, CD3-zeta, CD3 epsilon, CD5, CD22, CD79a, CD79b, and CD66d. The CD-19 binding domain can be a single chain antibody or single chain antibody fragment, such as an scFv.

In some embodiments, the anti-CD19 binding domain comprises a light chain variable region comprising the amino acid sequence set forth in SEQ ID NO: 1 and/or a heavy chain variable region comprising the amino acid sequence set forth in SEQ ID NO: 2. In another embodiment, the CD-19 CAR can include an anti-CD19 binding domain, a CD28 transmembrane domain (an exemplary CD28 transmembrane sequence is shown in SEQ ID NO: 3, a CD3z signaling domain (an exemplary CD3z sequence is shown in SEQ ID NO: 4 and can further include a suicide switch such as iCaspase9 and/or IL-15.

In some embodiments, the genetically engineered cord blood NK cells include a nucleic acid molecule encoding the heavy chain variable region of an anti-CD19 binding domain and/or a nucleic acid molecule encoding the light chain variable region of an anti-CD19 binding domain.

In some embodiments, the genetically engineered cord blood NK cells are present at a concentration of between 6 M/mL to 120 M/mL in pharmaceutical compositions and formulations. In some embodiments, the genetically engineered cord blood NK cells are present at a concentration of between 6 M/mL to 200 M/mL. In some embodiments, the genetically engineered cord blood NK cells are present at a concentration of between 6 M/mL to 25 M/mL. In some embodiments, the genetically engineered cord blood NK cells are present at a concentration of between 6 M/mL to 120 M/mL in a volume of medium ranging from 30-45 mL. In some embodiments, the genetically engineered cord blood NK cells are present at a concentration of between 6 M/mL to 200 M/mL in a volume of medium ranging from 30-45 mL. In some embodiments, the genetically engineered cord blood NK cells are present at a concentration of between 6 M/mL to 25 M/mL in a volume of medium ranging from 30-45 mL.

In some embodiments, CAR-NK cells are formulated at a concentration ranging from 100 million cells to 900 million cells, present in a volume of medium ranging from 30-45 mL. In some embodiments, CAR-NK cells are present at a concentration of about 200 million cells in a volume of about 36 mL. In another embodiment, CAR-NK cells are present at a concentration of about 800 million cells in a volume of about 36 mL.

In some embodiments, the NK cells are freshly isolated or from a cell line.

In some embodiments, the NK cells are derived from cord-blood, peripheral blood, T cells, iPS cells. In some embodiments, the NK cells are derived from cord-blood. In some embodiments, the NK cells are derived from peripheral blood. In some embodiments, the NK cells are derived from T cells. In some embodiments, the NK cells are derived from iPS cells.

In some embodiments, the NK cells are derived from cord-blood.

In certain embodiments, the NK cells comprise human cord blood-derived NK cells (CB-NK) transduced with a retroviral vector expressing an iCaspase9, a CD19-CAR and an IL-15. In a particular embodiment, the NK cells comprise human cord blood-derived NK cells (CB-NK) transduced with a non-retroviral vector expressing an iCaspase9, a CD19-CAR and an IL-15.

In some embodiments, the CD19-directed genetically modified NK cell immunotherapy (e.g., CD19 CAR+ viable NK cells and/or allogenic cord blood derived CD19 CAR NK⁺ cells) comprises cells that are genetically engineered cord blood NK cells comprising a CD19-CAR comprising a light chain variable region comprising the amino acid sequence set forth in SEQ ID NO: 1 and/or a heavy chain variable region comprising the amino acid sequence set forth in SEQ ID NO: 2. In another embodiment, the CD-19 CAR can include an anti-CD19 binding domain, a CD28 transmembrane domain (an exemplary CD28 transmembrane sequence is shown in SEQ ID NO: 3, a CD3z signaling domain (an exemplary CD3z sequence is shown in SEQ ID NO: 4 and can further include a suicide switch such as iCaspase9 and/or IL-15. In one embodiment, the genetically engineered cord blood NK cells include a nucleic acid molecule encoding the heavy chain variable region of an anti-CD19 binding domain set forth in SEQ ID NO: 2 and/or a nucleic acid molecule encoding the light chain variable region of an anti-CD19 binding domain set forth in SEQ ID NO: 1.

In one embodiment, the genetically engineered cord blood NK cells include a nucleic acid encoding the heavy chain variable region of an anti-CD19 binding domain set forth in SEQ ID NO: 2 and the light chain variable region of an anti-CD19 binding domain set forth in SEQ ID NO: 1. In some embodiments, the genetically engineered cord blood NK cells include a nucleic acid encoding the heavy chain variable region of an anti-CD19 binding domain comprising SEQ ID NO: 2, a light chain variable region of an anti-CD19 binding domain comprising SEQ ID NO: 1, and a CD28 transmembrane domain comprising SEQ ID NO: 3, a CD3z signaling domain comprising SEQ ID NO: 4.

In some embodiments, the genetically engineered cord blood NK cells include a nucleic acid encoding the heavy chain variable region of an anti-CD19 binding domain comprising SEQ ID NO: 2, a light chain variable region of an anti-CD19 binding domain comprising SEQ ID NO: 1, a CD28 transmembrane domain comprising SEQ ID NO: 3 or functional fragment thereof, a CD3z signaling domain comprising SEQ ID NO: 4 or functional fragment thereof and an IgG1 domain comprising SEQ ID NO: 5 or functional fragment thereof.

In some embodiments, the genetically engineered cord blood NK cells include a nucleic acid encoding the heavy chain variable region of an anti-CD19 binding domain comprising SEQ ID NO: 2, a light chain variable region of an anti-CD19 binding domain comprising SEQ ID NO: 1, a CD28 transmembrane domain comprising SEQ ID NO: 3, a CD3z signaling domain comprising SEQ ID NO: 4 and an IgG1 domain comprising SEQ ID NO: 5.

In some embodiments, the genetically engineered cord blood NK cells include a nucleic acid encoding the heavy chain variable region of an anti-CD19 binding domain comprising SEQ ID NO: 2, a light chain variable region of an anti-CD19 binding domain comprising SEQ ID NO: 1, a CD28 transmembrane domain comprising SEQ ID NO: 3 or functional fragment thereof, a CD3z signaling domain comprising SEQ ID NO: 4 or functional fragment thereof, an IgG1 domain comprising SEQ ID NO: 5 or functional fragment thereof, an IL15 comprising SEQ ID NO: 6 or functional fragment thereof, and an iCaspase 9 comprising SEQ ID NO: 7 or functional fragment thereof.

In some embodiments, the genetically engineered cord blood NK cells include a nucleic acid encoding an anti-CD19 binding domain heavy chain variable region comprising SEQ ID NO: 2, an anti-CD19 binding domain light chain variable region comprising SEQ ID NO: 1, a CD28 transmembrane domain comprising SEQ ID NO: 3, a CD3z signaling domain comprising SEQ ID NO: 4, an IgG1 domain comprising SEQ ID NO: 5, an IL15 comprising SEQ ID NO: 6, and an iCaspase 9 comprising SEQ ID NO: 7.

In some aspects, a cell therapy product (e.g., a CAR-NK cell therapy product) is provided for administration to a subject in need thereof comprising: (a) a population of engineered NK cells comprising cord blood NK cells transduced with a retroviral vector expressing anti-CD19 chimeric antigen receptor (CAR), IL-15, and iCaspase9; and (b) a pharmaceutically acceptable carrier as described herein.

In some embodiments, a cell therapy product suitable for administration to a subject in need thereof comprises a population of CAR-NK cells expressing anti-CD19 chimeric antigen receptor (CAR), IL-15, and iCaspase9 formulated in a medium comprising a cryoprotectant, a disaccharide, an albumin and a non-pyrogenic and isotonic crystalloid solution, wherein the population of cells comprises 200 million CAR-NK cells to 800 million CAR-NK cells. In a particular embodiment, a cell therapy product suitable for administration to a subject in need thereof comprises 200-800 million CAR-NK cells expressing anti-CD19 chimeric antigen receptor (CAR), IL-15, and iCaspase9 formulated in a medium for cryopreservation.

In some embodiments, the CD19-CAR NK cells comprise IL-15. In some embodiments, the IL-15 comprises SEQ ID NO: 6 or a functional fragment thereof. In some embodiments, the IL-15 comprises SEQ ID NO: 6.

In some embodiments, the CD19-CAR NK cells comprise iCaspase9. In some embodiments, the iCaspase9 comprises SEQ ID NO: 7 or a functional fragment thereof. In some embodiments, the iCaspase9 comprises SEQ ID NO: 7.

In some aspects, a cell therapy product is provided comprising a population of CAR-NK cell comprising cord blood NK cells genetically modified to express a CD-19 CAR, an iCaspase and an IL-15 formulated in a medium for cryopreservation.

In some embodiments, the concentration of cells in the product is between about 6 million cells/mL and 200 million cells/mL. In some embodiments, the concentration of cells in the product is between about 6 million cells/mL and 120 million cells/mL.

In some embodiments, the total viable cells post thawing is between about 200 million to about 800 million cells. In some embodiments, the total viable cells post thawing is about 200 million cells. In some embodiments, the total viable cells post thawing is about 300 million cells. In some embodiments, the total viable cells post thawing is about 400 million cells. In some embodiments, the total viable cells post thawing is about 500 million cells. In some embodiments, the total viable cells post thawing is about 600 million cells. In some embodiments, the total viable cells post thawing is about 700 million cells. In some embodiments, the total viable cells post thawing is about 800 million cells.

In one aspect, the present invention provides a method of treating cancer in an individual, comprising a step of administering a therapeutically effective amount of CD19-CAR+ viable cord blood natural killer (CB-NK) cells to the individual, wherein the cancer is CD19 negative.

In some embodiments, the cancer is a solid tumor. In some embodiments, the cancer is not a solid tumor.

In some embodiments, the cancer is a non-solid tumor.

In some embodiments, the cancer is large B-cell lymphoma.

In some embodiments, the CD19-CAR+ viable CB-NK cells are administered at a dose of at least 200×10⁶.

Various aspects of the invention are described in detail in the following sections. The use of sections is not meant to limit the invention. Each section can apply to any aspect of the invention. In this application, the use of “or” means “and/or” unless stated otherwise. As used herein, the singular forms “a”, “an”, and “the” include both singular and plural referents unless the context clearly dictates otherwise.

### Definitions

Administering: As used herein, the terms “administering,” or “introducing” are used interchangeably in the context of delivering a CD19-directed genetically modified NK cell immunotherapy described herein (e.g., CD19 CAR+ viable NK cells and/or allogenic cord blood derived CD19 CAR NK+ cells) to a patient in need thereof. Various methods are known in the art for administering cells to patients, including for example administering the cells to a patient in need thereof by intravenous or surgical methods.

Adoptive Cell Therapy: As used herein interchangeably, the terms “adoptive cell therapy” or “adoptive cell transfer” or “cell therapy” or “ACT” refer to the transfer of cells, for example, a population of genetically modified cells, into a patient in need thereof. The cells can be derived and propagated from the patient in need thereof (i.e., autologous cells) or could have been obtained from a non-patient donor (i.e., allogeneic cells). In some embodiments, the cell is an immune cell, such as a lymphocyte. In some embodiments, the immune cell is a NK cell. Various cell types can be used for ACT including but not limited to, natural killer (NK) cells, T cells, CD8+ cells, CD4+ cells, delta-gamma T-cells, regulatory T-cells, induced pluripotent stem cells (iPSCs), iPSC derived T cells, iPSC derived NK cells, hematopoietic stem cells (HSCs), mesenchymal stem cells (MSCs) and peripheral blood mononuclear cells.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

Approximately or about: As used herein, the term “approximately” or “about,” as applied to one or more values of interest, refers to a stated value of interest as well as value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Allogeneic: As used herein, allogeneic refers to any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenically

Autologous: As used herein, the term “autologous” means from the same individual. For example, “autologous” in relation to donor and recipient means that the donor subject is the recipient subject.

Chimeric Antigen Receptor (CAR): As used herein, the term “chimeric antigen receptor” or “CAR” engineered receptors which can confer an antigen specificity onto cells (for example, immune cells such as NK cells including cord blood derived NK cells and iPSC derived NK cells (iNK cells)). CARs are also known as chimeric antigen receptors or chimeric immunoreceptors. In various embodiments, a CAR described herein may include one or more of an antigen-specific targeting domain, an extracellular domain, a transmembrane domain, optionally one or more co-stimulatory domains, and an intracellular signaling domain.

CD19-directed genetically modified NK cell immunotherapy: As used herein, the term “CD19-directed genetically modified NK cell immunotherapy” refers to compositions and formulations comprising CD19 CAR+NK cells. In some embodiments, the CD19-directed genetically modified NK cell immunotherapy comprises CD19 CAR+ viable NK cells. In some embodiments, the CD19-directed genetically modified NK cell immunotherapy comprises cord blood derived CD19 CAR NK⁺ cells. In some embodiments, the CD19-directed genetically modified NK cell immunotherapy comprises allogenic cord blood derived CD19 CAR NK⁺ cells.

Cells: As used herein, the term “cells” refers to any cell unless a specific type of cell is named. In some embodiments, the cells are a stem cell or progenitor cell. In certain embodiments, the cells are somatic cells, e.g., adult stem cell, progenitor cell, or differentiated cell. In some embodiments, the cells are hematopoietic cell, e.g., a hematopoietic stem or progenitor cell. In some embodiments, the cells include B-cells, T cells, monocytes or progenitor cells. In some embodiments, the cells are NK cells, and in particular CAR-NK cells.

Cryoprotectant: As used herein, the term “cryoprotectant” means a substance used to protect biological tissue from freezing damage. Exemplary cryoprotectants include, for example, dimethyl sulfoxide (DMSO), glycerol, ethylene glycol and propanediol.

Engineered: As used herein, the term “engineered” refers to an entity that is generated by the hand of man, including a cell, nucleic acid, polypeptide, vector, and so forth. In at least some cases, an engineered entity is synthetic and comprises elements that are not naturally present or configured in the manner in which it is utilized in the disclosure.

Exogenous: As used herein, the “exogenous” as used herein refers to a polynucleotide (such as one encoding a gene product or part of a gene product) that is not present endogenously in a mammalian cell, such as an immune cell, or is synthetically generated outside of a mammalian cell, such as by recombinant technology. In a specific case, a particular gene product may be provided to a cell exogenously, and the cell may or may not also express the corresponding endogenous gene product in the cell.

Ex vivo: As used herein, the term “ex vivo” means a process in which cells are removed from a living organism and are propagated outside the organism (e.g., in a test tube, in a culture bag, in a bioreactor).

Fresh cell or Rescued Fresh Cell: As used herein, the terms “fresh,” “fresh cell,” or “rescued fresh cell” refers to mammalian cells that have never been frozen and/or once frozen but subsequently restimulated, cultured in culture medium and then harvested as fresh cells.

Functional equivalent or derivative: As used herein, the term “functional equivalent” or “functional derivative” denotes, in the context of a functional derivative of an amino acid sequence or any other molecule (e.g., a media formulation component) that retains an activity (either function or structural) that is substantially similar to that of the original molecule or sequence. A functional derivative or equivalent may be a natural derivative or is prepared synthetically. Exemplary derivatives include those having chemico-physical properties which are similar to that of the original molecule or sequence. Desirable similar chemico-physical properties include, similarities in charge, bulkiness, hydrophobicity, hydrophilicity, and the like.

Isotonic: As used herein, the term “isotonic” means having an osmotic pressure that is equal to or approximately the same as the osmotic pressure of a physiological fluid.

In vitro: As used herein, the term “in vitro” refers to events that occur in an artificial environment, e.g., in a test tube or reaction vessel, in cell culture, etc., rather than within a multi-cellular organism.

In vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Primary Cell: The term, “primary cell,” refers to cells that are directly isolated from a subject and which are subsequently propagated.

Polypeptide: The term, “polypeptide,” as used herein refers a sequential chain of amino acids linked together via peptide bonds. The term is used to refer to an amino acid chain of any length, but one of ordinary skill in the art will understand that the term is not limited to lengthy chains and can refer to a minimal chain comprising two amino acids linked together via a peptide bond. As is known to those skilled in the art, polypeptides may be processed and/or modified.

Protein: The term “protein” as used herein refers to one or more polypeptides that function as a discrete unit. If a single polypeptide is the discrete functioning unit and does not require permanent or temporary physical association with other polypeptides in order to form the discrete functioning unit, the terms “polypeptide” and “protein” may be used interchangeably. If the discrete functional unit is comprised of more than one polypeptide that physically associate with one another, the term “protein” refers to the multiple polypeptides that are physically coupled and function together as the discrete unit.

Subject: As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, and/or condition has been diagnosed with or displays one or more symptoms of the disease, disorder, and/or condition.

Sugar or Saccharide: The terms “sugar” and “saccharide” herein have been used interchangeably, and generally refer to oligosaccharides such as monosaccharides, disaccharides, trisaccharides or polysaccharides, and the like. In some embodiments, the saccharide is one or more of glucose, xylose, arabinose, fructose, galactose, mannose, mannitol, sorbitol, xylitol, myoinositol, trehalose, sucrose, lactose, maltose, cellobiose, lactitol, maltitol, methyl cellulose, carboxymethyl cellulose, dextran, glycogen, amylose, amylopectin, inulin, sodium alginate, ethyl cellulose, hydroxyethyl cellulose, raffinose, stachyose, xanthan gum, glucosamine, and galactosamine. In some embodiments, saccharide is a disaccharide. In some embodiments, the disaccharide is sucrose, lactose, maltose, trehalose, cellobiose, or chitobiose. In some other embodiments, the disaccharide is trehalose. In some embodiments, one or more sugars includes trehalose, sucrose, mannitol, and/or dextran.

Therapeutically effective amount: As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition. It will be appreciated by those of ordinary skill in the art that a therapeutically effective amount is typically administered via a dosing regimen comprising at least one unit dose. In some embodiments, a therapeutically effective amount of an adoptive cell therapy, as used herein, is a dosage of cells (e.g., a population of genetically modified immune cells such as CAR-T or CAR-NK) in a certain formulation (e.g., a cryopreservation media described herein) administered to a subject in need thereof (e.g., a patient suffering from a B-cell malignancy). For example, in some embodiments, a therapeutically effective amount comprises CAR-NK cells at a concentration of between 6 M/mL to 120 M/mL in a volume between 10 mL and 45 mL. In some embodiments, a therapeutically effective amount comprises CAR-NK cells at a concentration of between 5M/mL to 25M/mL in a volume between 10 mL and 45 mL. In some embodiments, a therapeutically effective amount comprises CAR-NK cells at an amount of about 200 million cells to about 800 million cells. In a particular embodiment, the CAR-NK cells have been genetically modified to express an iCaspase, an IL-15 and a CD-19 chimeric antigen receptor.

In a particular embodiment, the CAR-NK cells have been genetically modified to express a CD-19 CAR comprising a heavy chain variable region comprising the amino acid sequence set forth in SEQ ID NO:2 or a sequence having at least 95% identity to the sequence set forth in SEQ ID NO: 2 and/or a light chain variable region comprising the amino acid sequence set forth in SEQ ID NO: 1 or a sequence having at least 95% identity to the amino acid sequence set forth in SEQ ID NO: 1.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

The recitation of numerical ranges by endpoints herein includes all numbers and fractions subsumed within that range (e.g., 1 to 5 includes 1, 1.5, 2, 2.75, 3, 3.9, 4 and 5). It is also to be understood that all numbers and fractions thereof are presumed to be modified by the term “about.”

### BRIEF DESCRIPTION OF THE DRAWINGS

Drawings are for illustration purposes only; not for limitation.

FIG. 1A-1B are exemplary graphs demonstrating CAR-NK cytotoxicity (% killing) against Raji-CD19 KO cells (FIG. 1A) and Nalm6-CD19 KO cells (FIG. 1B).

### DETAILED DESCRIPTION

Eligible treatment options for patients who relapse following anti-CD19 treatment are limited and there exists a need for effective therapies for this relapsed population. There are two distinct types of patients that relapse including CD19-positive relapse and CD19-negative relapse. In patients with CD19-positive relapse, CD19 is still present on the surface of tumor cells but the anti-CD 19 therapy has limited persistence and anti-CD19 T cell CAR therapy have low potency. For CD19-negative relapse, CD19 is absent on the tumor cells, resulting in ineffective CAR T cell therapy as the tumors evade CAR-mediated recognition and clearance, in spite of CAR T-cell persistence. CD19 CAR+NK cells are advantageous compared to currently available CD19 CAR-T products and developing CD19 CAR-T cells (e.g., iCAR-T), since NK cells have capability of innate immune receptor-mediated killing. CD19 CAR+NK cells derived from cord blood cells do not require HLA selection (See, e.g., Liu et al. Use of CAR-transduced Natural Killer Cells in CD19 Positive Lymphoid Tumors. NEJM 382 (2020) 545-). Therefore, CD19 CAR+CB-NK cells described herein for patients with a history of anti-CD19-targeted therapy are advantageous compared with allogenic CAR-T cells.

In some embodiments, individuals with a history of anti-CD19 therapy (e.g., patients who have undergone CAR-T therapy or anti-CD19 monoclonal antibodies) are allowed sufficient time to recover from the previous anti-CD19 therapy prior to administration of CAR NK cells of the present invention. In some embodiments, the previously received anti-CD19-targeted therapy was CAR-T therapy. In some embodiments, the previously received anti-CD19-targeted therapy was a monoclonal antibody or an antibody drug conjugate.

In some embodiments, the individual received anti-CD19 targeted therapy within 5 years, within 4 years, within 3 years, within 2 years, within 1 year, within 11 months, within 10 months, within 9 months, within 8 months, within 7 months, within 6 months, within 5 months, within 4 months, within 3 months, within 2 months, within 1 month, within 4 weeks, within 3 weeks, or within 2 weeks prior to administration of CD19 CAR+CB-NK cells described herein.

In some embodiments, the individual received anti-CD19 targeted therapy at least 1 month, at least 2 months, at least 3 months, at least 4 months, at least 5 months, at least 6 months, at least 7 months, at least 8 months, at least 9 months, at least 10 months, at least 11 months, at least 12 months, at least 14 months, at least 16 months, at least 18 months, at least 20 months, at least 22 months, at least 24 months, at least 36 months prior to administration of CD19 CAR+CB-NK cells described herein.

In some embodiments, the individual received anti-CD19 targeted therapy at least 3 months prior to administration of CAR NK cells described herein. In some embodiments, individuals received anti-CD19 targeted therapy at least 6 months prior to administration of CAR NK cells described herein.

In some embodiments, individuals received anti-CD19 targeted therapy at least 1 week, at least 2 weeks, least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 16 weeks, at least 20 weeks, at least 24 weeks, at least 28 weeks or at least 32 weeks prior to administration of CAR NK cells described herein. In some embodiments, individuals received anti-CD19 therapy at least 12 weeks prior to administration of CAR NK cells described herein. In some embodiments, individuals received anti-CD19 targeted therapy at least 2 weeks prior to administration of CAR CB-NK cells described herein.

In some embodiments, the individual has a minimum life expectancy ≥12 weeks.

In some embodiments, the individual has an Eastern Cooperative Oncology Group (ECOG) performance status of 0 or 1.

In some embodiments, the individual has a cancer that expresses CD19 (e.g., CD19 positive (CD19+)). In some embodiments, the individual has a cancer that is CD19 negative (CD19-).

### CAR-NK Cell Compositions and Formulations

Provided herein are pharmaceutical compositions and formulations comprising CD19-directed genetically modified NK cell immunotherapy (e.g., CD19 CAR+ viable NK cells and/or “allogenic cord blood derived CD19 CAR NK⁺ cells). CD19 CAR+NK cells described herein comprise CD19 CAR, IL-15, and iCaspase9 (inducible caspase 9). In some embodiments, CD19 CAR+NK cells described herein comprise an exogenous gene encoding a CD19 CAR (e.g., a polypeptide comprising SEQ ID Nos 1-5), IL-15, and iCaspase9 (inducible caspase 9). In some embodiments, the IL-15 amino acid sequence comprises SEQ ID NO: 6. In some embodiments, the iCaspace9 amino acid sequence comprises SEQ ID NO: 7.

In some embodiments, CD19 CAR+NK cells are genetically engineered cord blood NK cells including a CD19-CAR comprising an anti-CD19 binding domain comprising a light chain variable region comprising the amino acid sequence set for the in SEQ ID NO:1 and a heavy chain variable region comprising the amino acid sequence set forth in SEQ ID NO: 2. In some embodiments, CD19 CAR+NK cells are genetically engineered cord blood NK cells including a CD19-CAR comprising an anti-CD19 binding domain comprising a light chain CDR1, CDR2 and CDR3 of SEQ ID NO:1 and a heavy chain CDR1, CDR2 and CDR3 SEQ ID NO: 2.

In some embodiments, the CD19 CAR+CB-NK cells comprise a CD28 domain. In some embodiments, the CD28 domain comprises SEQ ID NO: 3. In some embodiments, the CD19 CAR+CB-NK cells comprise a CD32 domain. In some embodiments, the CD32 domain comprises SEQ ID NO: 4.

Anti-CD19 Light chain variable fragment, VL:
(SEQ ID NO: 1)
DIQMTQTTSSLSASLGDRVTISCRASQDISKYLNWYQQKPDGTVK
LLIYHTSRLHSGVPSRFSGSGSGTDYSLTISNLEQEDIATYFCQQ
GNTLPYTFGGGTKLELKR
Anti-CD19 Heavy chain variable fragment, VH:
(SEQ ID NO: 2)
EVQLQQSGPGLVAPSQSLSVTCTVSGVSLPDYGVSWIRQPPRKGL
EWLGVIWGSETTYYNSALKSRLTIIKDNSKSQVFLKMNSLQTDDT
AIYYCAKHYYYGGSYAMDYWGQGTTVTVSSYVTVSSQDPA
CD28:
(SEQ ID NO: 3)
FWVLVVVGGVLACYSLLVTVAFIIFWVRSKRSRLLHSDYMNMTPR
RPGPTRKHYQPYAPPRDFAAYRS
CD35:
(SEQ ID NO: 4)
RVKFSRSADAPAYQQGQNQLYNELNLGRREEYDVLDKRRGRDPEM
GGKPRRKNPQEGLYNELQKDKMAEAYSEIGMKGERRRGKGHDGLY
QGLSTATKDTYDALHMQALPPRGP
IgG1:
(SEQ ID NO: 5)
EPKSPDKTHTCPPCPAPELLGGPSVFLFPPKPKDTLMISRTPEVT
CVVVDVSHEDPEVKFNWYVDGVEVHNAKTKPREEQYNSTYRVVSV
LTVLHQDWLNGKEYKCKVSNKALPAPIEKTISKAKGQPREPQVYT
LPPSRDELTKNQVSLTCLVKGFYPSDIAVEWESNGQPENNYKTTP
PVLDSDGSFFLYSKLTVDKSRWQQGNVFSCSVMHEALHNHYTQKS
LSLSPGKKDPK
IL-15:
(SEQ ID NO: 6)
MRISKPHLRSISIQCYLCLLLNSHFLTEAGIHVFILGCFSAGLPK
TEANWVNVISDLKKIEDLIQSMHIDATLYTESDVHPSCKVTAMKC
FLLELQVISLESGDASIHDTVENLIILANNSLSSNGNVTESGCKE
CEELEEKNIKEFLQSFVHIVQMFINTS
iCasp9:
(SEQ ID NO: 7)
MLEGVQVETISPGDGRTFPKRGQTCVVHYTGMLEDGKKVDSSRDR
NKPFKFMLGKQEVIRGWEEGVAQMSVGQRAKLTISPDYAYGATGH
PGIIPPHATLVFDVELLKLESGGGSGVDGFGDVGALESLRGNADL
AYILSMEPCGHCLIINNVNFCRESGLRTRTGSNIDCEKLRRRFSS
LHFMVEVKGDLTAKKMVLALLELAQQDHGALDCCVVVILSHGCQA
SHLQFPGAVYGTDGCPVSVEKIVNIFNGTSCPSLGGKPKLFFIQA
CGGEQKDHGFEVASTSPEDESPGSNPEPDATPFQEGLRTFDQLDA
ISSLPTPSDIFVSYSTFPGFVSWRDPKSGSWYVETLDDIFEQWAH
SEDLQSLLLRVANAVSVKGIYKQMPGCFNFLRKKLFFKTSASRA

In some embodiments, the NK cells are from primary cell isolates (e.g., NK cell derived from cord blood). In some embodiments, the NK cells are from a cell line. In some embodiments, the NK cells are fresh cells. In some embodiments, the NK cells were previously frozen and thawed.

In some embodiments, the NK cells are engineered to express one or more cytokines. In some embodiments, the NK cells are engineered to express one or more of IL-15, complex of IL-15 and IL-15Ra, IL-18, IL-12, IL-7, CCL19. Accordingly, in some embodiments, the NK cells are engineered to express IL-15. In some embodiments, the NK cells are engineered to express a complex of IL-15 and IL-15Ra. In some embodiments, the NK cells are engineered to express IL-18. In some embodiments, the NK cells are engineered to express IL-12. In some embodiments, the NK cells are engineered to express IL-7. In some embodiments, the NK cells are engineered to express CCL19.

In some embodiments, the NK cells are engineered to express one or more suicide genes. For example, in some examples the NK cells are engineered to express one or more of iCaspase9, non-secretable TNFalpha, herpes simplex virus thymidine kinase (HSV-TK), Uracil phosphoribosyl transferase (UPRTase), Cytosine deaminase (CD).

Additional examples of suicide genes include engineered nonsecretable (including membrane bound) tumor necrosis factor (TNF)-alpha mutant polypeptides (see e.g., PCT/US2019/062009, which is incorporated by reference herein in its entirety), and they may be affected by delivery of an antibody that binds the TNF-alpha mutant. Examples of suicide gene/prodrug combinations that may be used are Herpes Simplex Virus-thymidine kinase (HSV-tk) and ganciclovir, acyclovir, or FIAU; oxidoreductase and cycloheximide; cytosine deaminase and 5-fluorocytosine; thymidine kinase thymidilate kinase (Tdk::Tmk) and AZT; and deoxycytidine kinase and cytosine arabinoside. The E. coli purine nucleoside phosphorylase, a so-called suicide gene that converts the prodrug 6-methylpurine deoxyriboside to toxic purine 6-methylpurine, may be utilized. Other suicide genes include CD20, CD52, inducible caspase 9, purine nucleoside phosphorylase (PNP), Cytochrome p450 enzymes (CYP), Carboxypeptidases (CP), Carboxylesterase (CE), Nitroreductase (NTR), Guanine Ribosyltransferase (XGRTP), Glycosidase enzymes, Methionine-a, g-lyase (MET), and Thymidine phosphorylase (TP), as examples.

In some embodiments, the NK cells are engineered to express one or more of iCaspase9. In some embodiments, the NK cells are engineered to express non-secretable TNFalpha. In some embodiments, the NK cells are engineered to express herpes simplex virus thymidine kinase (HSV-TK). In some embodiments, the NK cells are engineered to express Uracil phosphoribosyl transferase (UPRTase). In some embodiments, the NK cells are engineered to express Cytosine deaminase (CD).

In some embodiments the NK cell is gene edited to allow the cells to work more effectively in a tumor microenvironment. In some embodiments, the genes are one or more of TDAG8, NKG2A, SIGLEC-7, LAG3, TIM3, CISH, FOXO1, TGFBR2, TIGIT, CD96, ADORA2, NR3C1, PD1, PDL-1, PDL-2, CD47, SIRPA, SHIP1, ADAM 17, RPS6, 4EBP1, CD25, CD40, IL21R, ICAM1, CD95, CD80, CD86, IL10R, CD5, and CD7. In some embodiments, one or more of these genes are knocked out or knocked down in the cells.

In some embodiments, the NK cells are engineered to express CD19-CAR, IL-15, and iCaspase9. An exemplary CAR-NK cell comprising CD19 IL-15, and iCaspase9 is described in Leukemia 32 (2018) 520-531, incorporated herein by reference in its entirety.

In some embodiments, the genetically engineered cord blood NK cells include a CD19-CAR comprising an anti-CD19 binding domain, a transmembrane domain such as the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154 and an intracellular signaling domain such as an intracellular signaling domain FcR gamma, FcR beta, CD3 gamma, CD3 delta, CD3-zeta, CD3 epsilon, CD5, CD22, CD79a, CD79b, and CD66d. The CD-19 binding domain can be a single chain antibody or single chain antibody fragment, such as an scFv. In one embodiment, the anti-CD19 binding domain comprises a light chain variable region comprising the amino acid sequence set forth in SEQ ID NO: 1 and/or a heavy chain variable region comprising the amino acid sequence set forth in SEQ ID NO: 2. In another embodiment, the CD-19 CAR can include an anti-CD19 binding domain, a CD28 transmembrane domain (an exemplary CD28 transmembrane sequence is shown in SEQ ID NO: 3), a CD3z signaling domain (an exemplary CD3z sequence is shown in SEQ ID NO: 4) and can further include a suicide switch such as iCaspase9 and/or IL-15.

In one embodiment, the genetically engineered cord blood NK cells include a nucleic acid molecule encoding the heavy chain variable region of an anti-CD19 binding domain and/or a nucleic acid molecule encoding the light chain variable region of an anti-CD19 binding domain.

In one aspect, the compositions described herein comprise CD19-directed genetically modified NK cell immunotherapy (e.g., CD19 CAR+ viable CB-NK cells and/or “allogenic cord blood derived CD19 CAR NK⁺ cells) and a pharmaceutically acceptable carrier.

In some embodiments, compositions comprising CD19-directed genetically modified NK cell immunotherapy described herein comprises CAR-NK cells at a concentration of between 6 M/mL to 120 M/mL, 6 M/mL to 200 M/mL, 5 M/mL to 25 M/mL, 6 M/mL to 120 M/mL in a 36 mL volume or 5 M/mL to 25 M/mL in a 36 mL volume.

In some embodiments, the total volume of a composition comprising CD19-directed genetically modified NK cell immunotherapy in which the CAR-NK cells are suspended is between about 15 mL and 30 mL, about 30 mL and 45 mL, about 30 and 60 mL, or about 30 mL and 75 mL. In some embodiments, the total volume in which the NK cells are suspended is between about 15 mL and 30 mL. In some embodiments, the total volume in which the CAR-NK cells are suspended is between about 30 mL and 45 mL. In some embodiments, the total volume in which the CAR-NK cells are suspended is between about 30 mL and 60 mL. In some embodiments, the total volume in which the CAR-NK cells are suspended is between about 30 mL and 75 mL. In some embodiments, the total volume in which the CAR-NK cells are suspended is about 20 mL, 21 mL, 22 mL, 23 mL, 24 mL, 25 mL, 26 mL, 27 mL, 28 mL, 29 mL, 30 mL, 31 mL, 32 mL, 33 mL, 34 mL, 35 mL, 36 mL, 37 mL, 38 mL, 39 mL, 40 mL, 41 mL, 42 mL, 43 mL, 44 mL, 45 mL, 46 mL, 47 mL, 48 mL, 49 mL or 50 mL. In some embodiments, the total volume in which the CAR-NK cells are suspended is about 20 mL. In some embodiments, the total volume in which the CAR-NK cells are suspended is about 21 mL. In some embodiments, the total volume in which the CAR-NK cells are suspended is about 22 mL. In some embodiments, the total volume in which the CAR-NK cells are suspended is about 23 mL. In some embodiments, the total volume in which the CAR-NK cells are suspended is about 24 mL. In some embodiments, the total volume in which the CAR-NK cells are suspended is about 25 mL. In some embodiments, the total volume in which the CAR-NK cells are suspended is about 26 mL. In some embodiments, the total volume in which the CAR-NK cells are suspended is about 27 mL. In some embodiments, the total volume in which the CAR-NK cells are suspended is about 28 mL. In some embodiments, the total volume in which the CAR-NK cells are suspended is about 29 mL. In some embodiments, the total volume in which the CAR-NK cells are suspended is about 30 mL. In some embodiments, the total volume in which the CAR-NK cells are suspended is about 31 mL. In some embodiments, the total volume in which the CAR-NK cells are suspended is about 32 mL. In some embodiments, the total volume in which the CAR-NK cells are suspended is about 33 mL. In some embodiments, the total volume in which the CAR-NK cells are suspended is about 34 mL. In some embodiments, the total volume in which the CAR-NK cells are suspended is about 35 mL. In some embodiments, the total volume in which the CAR-NK cells are suspended is about 36 mL. In some embodiments, the total volume in which the CAR-NK cells are suspended is about 37 mL. In some embodiments, the total volume in which the CAR-NK cells are suspended is about 38 mL. In some embodiments, the total volume in which the CAR-NK cells are suspended is about 39 mL. In some embodiments, the total volume in which the CAR-NK cells are suspended is about 40 mL. In some embodiments, the total volume in which the CAR-NK cells are suspended is about 41 mL. In some embodiments, the total volume in which the CAR-NK cells are suspended is about 42 mL. In some embodiments, the total volume in which the CAR-NK cells are suspended is about 43 mL. In some embodiments, the total volume in which the CAR-NK cells are suspended is about 44 mL. In some embodiments, the total volume in which the CAR-NK cells are suspended is about 45 mL. In some embodiments, the total volume in which the CAR-NK cells are suspended is about 46 mL.

In some embodiments, a composition comprising CD19-directed genetically modified NK cell immunotherapy comprise CAR-NK cells at a concentration of about 200 million to 800 million cells per 36 mL. In some embodiments, the composition comprises CAR-NK cells at a concentration of between about 100-1000 million CAR-NK cells per a 36 mL fill volume. In some embodiments, the composition comprises CAR-NK cells at a concentration of between about 200-800 million cells per a 36 mL fill volume. In some embodiments, the composition comprises CAR-NK cells at a concentration of about 100 million cells per a 36 mL fill volume. In some embodiments, the composition comprises CAR-NK cells at a concentration of about 200 million cells per a 36 mL fill volume. In some embodiments, the composition comprises CAR-NK cells at a concentration of about 300 million cells per a 36 mL fill volume. In some embodiments, the composition comprises CAR-NK cells at a concentration of about 400 million cells per a 36 mL fill volume. In some embodiments, the composition comprises CAR-NK cells at a concentration of about 500 million cells per a 36 mL fill volume. In some embodiments, the composition comprises CAR-NK cells at a concentration of about 600 million cells per a 36 mL fill volume. In some embodiments, the composition comprises CAR-NK cells at a concentration of about 700 million cells per a 36 mL fill volume. In some embodiments, the composition comprises CAR-NK cells at a concentration of about 800 million cells per a 36 mL fill volume. In some embodiments, the composition comprises CAR-NK cells at a concentration of about 1000 million cells per a 36 mL fill volume.

In some embodiments, the CAR-NK cell therapy product is an allogeneic cell therapy product comprised of human cord blood-derived NK cells transduced with a retroviral vector expressing iCaspase9, CD-19 CAR and IL-15.

In some embodiments, the CAR-NK cell therapy product comprises a population of cells between 1×10⁶ to 5×10⁹ formulated in a cryopreservation media described herein. In some embodiments, the CAR-NK cell therapy product comprises a population of cells between 2×10⁶ to 800×10⁶.

In some embodiments, the CAR-NK cell therapy product is an allogeneic cell therapy product comprising 200×10⁶ to 800×10⁶ of human cord blood-derived NK cells transduced with a retroviral vector expressing iCaspase9, CD-19 CAR and IL-15 and formulated in 36 mLs of a medium for cryopreservation of Table 1 in a 50 mL AT vial. In some embodiments, the CAR-NK cell therapy product is formulated in Cryopreservation Media 1 of Table 1. In some embodiments, the CAR-NK cell therapy product is formulated in Cryopreservation Media 2 of Table 1. In some embodiments, the CAR-NK cell therapy product is formulated in Cryopreservation Media 3 of Table 1. In some embodiments, the CAR-NK cell therapy product is formulated in Cryopreservation Media 4 of Table 1. In some embodiments, the CAR-NK cell therapy product is formulated in Cryopreservation Media 5 of Table 1.

In some embodiments, the CAR-NK cell therapy product is formulated in Cryopreservation Media 9 of Table 1.

In some embodiments, the CAR-NK cell therapy product is an allogeneic cell therapy product comprised of 200×10⁶ to 800×10⁶ of viable human cord blood-derived NK cells transduced with a retroviral vector expressing iCaspase9, CD-19 CAR and IL-15 and formulated in 36 mLs of medium for cryopreservation in a 50 mL AT vial.

Pharmaceutical compositions and formulations as described herein can be prepared by mixing the active ingredients (such as the cells) having the desired degree of purity with one or more optional pharmaceutically acceptable carriers (Remington's Pharmaceutical Sciences 22^{nd} edition, 2012), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

In some embodiments, CD19 CAR CB-NK cells are suspended in a medium for cryopreservation. In some embodiments, CD19 CAR CB-NK cells are suspended in the cryopreservation media described in Table 1. To prepare the medium in Table 1, 25% w/v HSA (human serum albumin) and 400 mg/mL Trehalose solution are used as component.

TABLE 1
Cryopreservation Media

| Cryopreservation Media | Composition |
|---|---|
| 1 | 50% v/v PLASMA-LYTE-HEPES + 35% v/v |
| | Dextran/Dextrose + 10% v/v HSA + 5% v/v |
| | DMSO |
| 2 | 40% v/v PLASMA-LYTE A + 50% v/v |
| | CS10 + 10% v/v HSA |
| 3 | 50% v/v MEM-HEPES + 35% v/v |
| | Dextran/Dextrose + 10% v/v HSA + 5% v/v |
| | DMSO |
| 4 | 40% v/v MEM + 50% v/v CS10 + 10% v/v |
| | HSA |
| 5 | 38.6% v/v PLASMA-LYTE A + 50% v/v |
| | CS10 + 10% v/v HSA + 0.8% v/v AA + |
| | Vitamin (Baxter; 0.2% vial 1 and 0.4% vials |
| | 2) + 30 mM trehalose |
| 6 | 40% v/v MEM + 50% v/v CS10 + 10% v/v |
| | HSA + 1.5 mM Glutathione |
| 7 | 40% v/v PLASMA-LYTE A + 50% v/v |
| | CS10 + 10% v/v HSA + 1.5 mM Glutathione |
| 8 | 38.6% v/v PLASMA-LYTE A + 50% v/v |
| | CS10 + 10% v/v HSA + 0.8% v/v AA + 0.6% |
| | v/v Vitamin + 1.5 mM Glutathione |
| 9 | 40% PLASMA-LYTE A + 50% CS10 + 10% |
| | HSA + 30 mM trehalose |
| | (e.g., 37.7% v/v PLASMA-LYTE A + 50% v/v |
| | CS10 + 9.4% v/v HSA + 2.8% v/v trehalose) |

### Methods of Treatment

The CAR-NK cell compositions described herein are suitable for adoptive cell therapy. Adoptive cell therapies can be used to treat various diseases, including, for example, cancer. In certain embodiments, the CAR-NK cell compositions are useful for the treatment of a cancer or a tumor. In certain embodiments, the cancer comprises breast, heart, lung, small intestine, colon, spleen, kidney, bladder, head, neck, ovarian, prostate, brain, pancreatic, skin, bone, bone marrow, blood, thymus, uterine, testicular, and liver tumors. In some embodiments, the cancer is a blood cancer. In some embodiments, the cancer is not limited to CD19 and CD20 double positive cancer.

In some embodiments, the blood cancer is a B-cell malignancy (e.g., relapsed or refractory B-cell non-Hodgkin lymphoma (including large B-cell lymphoma and indolent non-Hodgkin lymphoma)). In some embodiments, the cancer is chronic lymphocytic leukemia (CLL). In some embodiments, the cancer is acute lymphoblastic leukemia (ALL). In some embodiments, the cancer is a histologically proven B-cell NHL, including LBCL and iNHL (FL and MZL), including the types defined by the World Health Organization (WHO). In some embodiments, the CAR-NK cell compositions described herein are suitable for relapsed or refractory cancer in the individual, wherein the individual has previously received a CD19 targeted therapy (including CD19 targeted CAR T therapy and CD19 targeted antibody therapy). In some embodiments, the relapsed or refractory cancer may be CD19 positive or CD19 negative.

In some embodiments, the cancer is a previously treated r/r histologically proven Cluster of Differentiation (CD) 19 expressing disease. In some embodiments, the cancer is LBCL, including the subtypes defined by the World Health Organization (WHO). In some embodiments, the cancer is Diffuse large B-cell lymphoma (DLBCL) not otherwise specified (NOS). In some embodiments, the cancer is High-grade B-cell lymphoma (HGBL) with MYC and BCL2 and/or BCL6 rearrangement. In some embodiments, the cancer is HGBL NOS without translocations.

In some embodiments, the cancer is DLBCL arising from iNHL. In some embodiments, the cancer is follicular lymphoma (FL). In some embodiments, the cancer is marginal zone lymphoma (MZL). In some embodiments, the cancer is T-cell/histiocyte-rich LBCL. In some embodiments, the cancer is DLBCL associated with chronic inflammation. In some embodiments, the cancer is Epstein-Barr virus-positive DLBCL-NOS. In some embodiments, the cancer is Primary cutaneous DLBCL, leg type. In some embodiments, the cancer is Primary mediastinal large B-cell lymphoma (PMBCL). In some embodiments, the cancer is FL Grade 3B.

In some embodiments, the cancer is iNHL. In some embodiments, the cancer is FL Grades 1, 2, 3A. In some embodiments, the cancer is MZL (nodal, extranodal, and splenic).

In some embodiments, the patient has measurable disease, defined as at least 1 lesion per the Lugano classification. In some embodiments, the lesions situated in a previously irradiated area are considered measurable if radiographic progression has been documented in such lesions following completion of radiation therapy. In some embodiments, the LBCL have positron emission tomography (PET) positive disease per the Lugano classification.

In some embodiments, the patient has a disease that is r/r after at least 2 prior lines of systemic therapy. In some embodiments, the patient with r/r LBCL has received an anti-CD20 monoclonal antibody (mAb) and an anthracycline containing chemotherapy regimen and failed or be ineligible for high-dose chemotherapy and autologous stem cell transplantation (ASCT).

In some embodiments, the patient with iNHL has received an anti-CD20 mAb and an alkylating agent (e.g., bendamustine or cyclophosphamide). In some embodiments, preinduction salvage chemotherapy and ASCT is considered 1 therapy. In some embodiments, the patient consolidation/maintenance therapy after a chemotherapy regimen (without intervening relapse) is considered 1 line of therapy with the preceding combination therapy. In some embodiments, maintenance antibody therapy is not considered a line of therapy. In some embodiments, the patient single-agent anti-CD20 mAb therapy is not considered a line of therapy.

In some embodiments, the patient has adequate bone marrow function. In some embodiments, adequate bone marrow function is defined as absolute neutrophil count >500/μL and/or platelet count of >50,000/μL at screening. In some embodiments, patients with transfusion-dependent thrombocytopenia are excluded.

In some embodiments, the patient has adequate renal, hepatic, cardiac, and pulmonary function. In some embodiments, adequate renal, hepatic, cardiac, and pulmonary function comprises one or more of:
a) Estimated glomerular filtration rate (GFR; Modification of Diet in Renal Disease equation [MDRD])≥30 mL/min;
b) Serum alanine aminotransferase/aspartate aminotransferase≤5 times the upper limit of normal range (ULN), as long as participant is asymptomatic;
c) Total bilirubin≤2 mg/dL. Participants with Gilbert's syndrome may have a bilirubin level >2×ULN, per discussion between the investigator and the medical monitor;
d) Left ventricular ejection fraction (LVEF)>40% as determined by an echocardiogram (ECHO) or multigated acquisition (MUGA) scan performed within 1 month of determination of eligibility;
e) No evidence of clinically relevant pericardial effusion, and no acute clinically significant electrocardiogram (ECG) findings;
f) Absence of Grade ≥2 pleural effusion. Grade 1 stable pleural effusions are allowed; and/or
g) Baseline oxygen saturation >92% on room air.

In some embodiments, the previously received CD19 targeted therapy is a CAR T therapy selected from the group consisting of axicabtagene ciloleucel, tisagenlecleucel, brexucabtagene autoleucel and or lisocabtagene maraleucel. In some embodiments, the previously administered CD19 targeted therapy is a CD19 targeted antibody therapy selected from the group consisting of tafasitamab and blinatumomab. In some embodiments, the previously administered CD19 targeted therapy is a CD19-targeted antibody-drug-conjugate. In some embodiments, the CD19-targeted antibody-drug-conjugate is loncastuximab tesirine.

In some embodiments, the subject is administered a composition comprising CAR-NK cells. In some embodiments, the CAR-NK cell comprises an anti-CD19 CAR gene and an IL-15 gene. In some embodiments, the CAR-NK cell comprises an anti-CD19 CAR gene, an IL-15 gene, and iCaspase9.

In some embodiments, the CAR-NK cells are frozen prior to administration. In some embodiments, the CAR-NK cells are not washed prior to administering to a subject in need thereof. In some embodiments, the CAR-NK cells are washed prior to administering to a subject in need thereof. In some embodiments, the frozen cells are thawed and administered into a patient in need thereof within about 30 minutes and 2 hours from thawing the cells. In some embodiments, the rate of intravenous infusion into a subject is between about 2-3 minutes.

In some embodiments, the adoptive cell therapy is used in combination with one or more additional cancer treatments, such as for example lymphodepleting chemotherapy. Accordingly, in some embodiments, a subject who has cancer receives lymphodepleting chemotherapy before administration of a CAR-NK cell therapy product formulated in a cryopreservation media described herein.

In some embodiments, the CAR-NK cell therapy is administered to a patient for treatment of a B-cell malignancy. In some embodiments, a CAR-NK cell therapy product comprises a CD19-CAR comprising an anti-CD19 binding domain, a transmembrane domain such as the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154 and an intracellular signaling domain such as an intracellular signaling domain FcR gamma, FcR beta, CD3 gamma, CD3 delta, CD3-zeta, CD3 epsilon, CD5, CD22, CD79a, CD79b, and CD66d. The CD-19 binding domain can be a single chain antibody or single chain antibody fragment, such as an scFv.

In one embodiment, the anti-CD19 binding domain comprises a light chain variable region comprising the amino acid sequence set forth in SEQ ID NO: 1 and/or a heavy chain variable region comprising the amino acid sequence set forth in SEQ ID NO: 2. In another embodiment, the CD-19 CAR can include an anti-CD19 binding domain, a CD28 transmembrane domain (an exemplary CD28 transmembrane sequence is shown in SEQ ID NO: 3, a CD3z signaling domain (an exemplary CD3z sequence is shown in SEQ ID NO: 4 and can further include a suicide switch such as iCaspase9 and/or IL-15.

In one embodiment, the CAR-NK cell therapy product comprises a nucleic acid molecule encoding the heavy chain variable region of an anti-CD19 binding domain and/or a nucleic acid molecule encoding the light chain variable region of an anti-CD19 binding domain.

### Concentrations and Volumes of CD19-CAR NK Cells

In some embodiments, the cell therapy product is a population of CD19-CAR NK cells that further comprise IL-15 and iCaspase9. In some embodiments, the cell therapy product comprises CD19-CAR NK cells at concentration of between about 6 and 120 million cells per milliliter. In some embodiments, the cell therapy product comprises CD19-CAR NK cells in a 50 mL container at concentration of between about 6 and 120 million cells per milliliter. In some embodiments, the cell therapy product comprises CD19-CAR NK cells in a 50 mL container at concentration of between about 3 and 150 million cells per milliliter. In some embodiments, the cell therapy product comprises CD19-CAR NK cells in a 50 mL container at concentration of between about 1 and 250 million cells per milliliter. In some embodiments, the cell therapy product comprises CD19-CAR NK cells in a 50 mL container at concentration of between about 1 and 350 million cells per milliliter. In some embodiments, the cell therapy product comprises CD19-CAR NK cells in a 50 mL container at a concentration of between about 1 and 500 million cells per milliliter.

In some embodiments, the cell therapy product comprises between about 20×10⁶ and 100×10⁷ cells in 50 mL container. In some embodiments, the cell therapy product comprises between about 100×10⁶ and 900×10⁶ cells in 50 mL container. In some embodiments, the cell therapy product comprises about 50×10⁶ cells in 50 mL container. In some embodiments, the cell therapy product comprises about 100×10⁶ cells in 50 mL container. In some embodiments, the cell therapy product comprises about 200×10⁶ cells in 50 mL container. In some embodiments, the cell therapy product comprises about 200×10⁶ cells in 50 mL container. In some embodiments, the cell therapy product comprises about 300×10⁶ cells in 50 mL container. In some embodiments, the cell therapy product comprises about 400×10⁶ cells in 50 mL container. In some embodiments, the cell therapy product comprises about 500×10⁶ cells in 50 mL container. In some embodiments, the cell therapy product comprises about 600×10⁶ cells in 50 mL container. In some embodiments, the cell therapy product comprises about 700×10⁶ cells in 50 mL container. In some embodiments, the cell therapy product comprises about 800×10⁶ cells in 50 mL container. In some embodiments, the cell therapy product comprises about 900×10⁶ cells in 50 mL container. In some embodiments, the cell therapy product comprises about 1000×10⁶ cells in 50 mL container. In some embodiments, the cell therapy product comprises about 1500×10⁶ cells in 50 mL container. In some embodiments, the cell therapy product comprises about 2000×10⁶ cells in 50 mL container. In some embodiments, the cell therapy product comprises about 2500×10⁶ cells in 50 mL container. In some embodiments, the cell therapy product comprises about 3000×10⁶ cells in 50 mL container. In some embodiments, the cell therapy product comprises about 3500×10⁶ cells in 50 mL container. In some embodiments, the cell therapy product comprises about 4000×10⁶ cells in 50 mL container. In some embodiments, the cell therapy product comprises about 4500×10⁶ cells in 50 mL container. In some embodiments, the cell therapy product comprises about 5000×10⁶ cells in 50 mL container.

In some embodiments, the cell therapy product is contained in a 50 mL container at a fill volume of about between 20-45 mL. In some embodiments, the cell therapy product is contained in a 50 mL container at a fill volume of about 36 mL. In some embodiments, the NK cell is engineered to comprise one or more transgenes, for example a chimeric antigen receptor (CAR). In some embodiments, the cells are CAR-NK+ cells. In some embodiments, the cell therapy product comprises a CD19-CAR, IL-15 transgene and an iCaspase9. In some embodiments, the cell therapy product comprises between about 100×10⁶ and 900×10⁶ CAR-NK+ cells in a 50-mL container. In some embodiments, the cell therapy product present is 200×10⁶ CAR-NK+ cells in a 50-mL container. In some embodiments, the cell therapy product present is 300×10⁶ CAR-NK+ cells in a 50-mL container. In some embodiments, the cell therapy product present is 400×10⁶ CAR-NK+ cells in a 50-mL container. In some embodiments, the cell therapy product present is 500×10⁶ CAR-NK+ cells in a 50-mL container. In some embodiments, the cell therapy product present is 600×10⁶ CAR-NK+ cells in a 50-mL container. In some embodiments, the cell therapy product present is 700×10⁶ CAR-NK+ cells in a 50-mL container. In some embodiments, the cell therapy product present is 800×10⁶ CAR-NK+ cells in a 50-mL container. In some embodiments, the cell therapy product present is 900×10⁶ CAR-NK+ cells in a 50-mL container.

### Viability Assessment

Viability of CAR-NK cells can be assessed in vitro using various methods known in the art. In some embodiments, the in vitro cell viability test includes the Trypan Blue exclusion assay. In some embodiments, other analytical methods can be used to assess the cell viability of cells, for example, flow cytometry-based viability markers and the like. A person of ordinary skill in the art can opt for any analytical method to assess the viability of CAR NK cells that can be applied to assess the cell viability cells described herein.

Phenotype and function of CAR-NK cells can be assessed in vitro using various methods known in the art. In some embodiments, the in vitro cell phenotyping tests includes flow cytometry assays. In some embodiments, the in vitro cell function test includes cytokine production, cytotoxicity, proliferation and other analytical methods.

Efficacy of CAR-NK cells in vivo can be assessed using animal studies known in the art. In some embodiments, the in vivo cell phenotyping tests immunodeficiency mice-based tumor models.

The CAR-NK cells described herein retain high viability (e.g., greater than 70%, 75%, 80%, 85%, 90%, 95%, or greater than 95%) and retain physiological characteristics of their native state, which allows the cells to be used for a variety of applications, such as for genetic manipulation of the cells, and for cell therapy purposes such as, for example, in adoptive cell therapy applications.

### Combination Therapy

In certain embodiments, the compositions and methods of the present embodiments involve an immune cell population (e.g., CAR NK cell population) in combination with at least one additional therapy. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, hormone therapy, oncolytic viruses, or a combination of the foregoing. The additional therapy may be in the form of adjuvant or neoadjuvant therapy.

In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemopreventative agent. The additional therapy may be one or more of the chemotherapeutic agents known in the art.

In particular embodiments, in addition to the inventive cell therapy of the disclosure, the individual may have been provided, may be provided, and/or will be provided a specific additional therapy for cancer, including one or more of surgery, radiation, immunotherapy (other than the cell therapy of the present disclosure), hormone therapy, gene therapy, chemotherapy, and so forth.

An immune cell therapy may be administered before, during, after, or in various combinations relative to an additional cancer therapy. The administrations may be in intervals ranging from concurrently to minutes to days to weeks. In embodiments where the immune cell therapy is provided to a patient separately from an additional therapeutic agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations, it may be desirable to extend the time period for treatment significantly where several days (2, 3, 4, 5, 6, or 7) to several weeks (1, 2, 3, 4, 5, 6, 7, or 8) lapse between respective administrations.

### EXAMPLES

Other features, objects, and advantages of the present invention are apparent in the examples that follow. It should be understood, however, that the examples, while indicating embodiments of the present invention, are given by way of illustration only, not limitation. Various changes and modifications within the scope of the invention will become apparent to those skilled in the art from the examples.

CAR-NK cells used in these examples comprised CD19 CAR, IL-15, and iCaspase9. Exemplary CAR-NK cells used in these examples were genetically engineered cord blood NK cells including a CD19-CAR comprising an anti-CD19 binding domain comprising a light chain variable region comprising the amino acid sequence set forth in SEQ ID NO: 1 and/or a heavy chain variable region comprising the amino acid sequence set forth in SEQ ID NO: 2. CAR-NK cells used herein are formulated in media for cryopreservation (e.g., a cryopreservation media described in Table 1).

### Example 1: Administration of CAR NK Cells to an Individual

This example describes exemplary patient population and administration of CAR NK cell to a cancer patient, such as a patient who has Relapsed or Refractory (r/r) B-cell Non-Hodgkin Lymphoma (NHL). Patients with a history of anti-CD19 therapy (e.g., CD19-targeted chimeric antigen receptor (CAR) T cells or monoclonal antibodies) are included. Patients with a history of anti-CD19 therapy (e.g., patients who have undergone CAR-T therapy) are allowed sufficient time to recover from the previous anti-CD19 therapy (e.g., at least 3 months) prior to administration of CAR NK cells described herein.

Patients meeting any of the following exclusion criteria are not enrolled in the study:
1. Patients with total body weight of <40 kg.
2. Patients with primary or secondary central nervous system (CNS) involvement by lymphoma. Patients with a history of secondary CNS involvement by lymphoma without evidence of CNS involvement at screening may be included.
3. Patients with Burkitt lymphoma, mantle cell lymphoma, lymphoplasmocytic lymphoma, or transformation from CLL/small lymphocytic lymphoma (Richter transformation).
4. Patients with a history of malignancy other than nonmelanoma skin cancer, carcinoma in situ (e.g., cervix, bladder, breast), low-grade tumors deemed to be cured and not treated with systemic therapy (e.g., by gastro-endoscopy curatively removed gastric cancer) or unless disease free for >3 years at screening.
5. Patients who have undergone autologous or allogeneic transplant or CAR-T therapy within 3 months of planned enrollment. Patients after allogeneic transplant have to be off systemic immunosuppressive therapy and without the evidence of clinically relevant acute or chronic GvHD at the time of enrollment.
6. Treatment with any investigational products or any systemic anticancer treatment within 14 days or 2 half-lives of the treatment (whichever is longer) before conditioning therapy.
7. Patients with clinical evidence of active infection, including fungal, bacterial, viral, or other infection that is uncontrolled or requires IV antimicrobials for management within 3 days before enrollment.
8. Patients with active HIV, HBV, or HCV, infection at screening (positive DNA/RNA test).
9. Patients with a history or presence of active or clinically relevant CNS disorder, such as seizure, encephalopathy, cerebrovascular ischemia/hemorrhage, severe dementia, cerebellar disease, or any autoimmune disease with CNS involvement. For CNS disorders that recover or are in remission, patients without recurrence within 2 years of planned study enrollment may be included.
10. Patients with any of the following within 6 months of enrollment: myocardial infarction, cardiac angioplasty or stenting, unstable angina, symptomatic congestive heart failure (i.e., New York Heart Association Class II or greater), clinically significant arrythmia (including uncontrolled atrial fibrillation), or any other clinically significant cardiac disease.
11. Patients who have received a live vaccine≤6 weeks before the start of the conditioning regimen

CD19 CAR-NK cells described herein are tested to evaluate the safety and tolerability in adult participants with r/r B-cell NHL. The study will include 2 parts: Part 1 (Dose escalation and dose expansion) and Part 2 with approximately 242 patients.

In Part 1, dose escalation and dose expansion cohort participants receive CD19 CAR-NK cells as follows:

Part 1: Dose escalation: CD19 CAR-NK cells-200×10⁶ CD19-CAR+ viable CB-NK cells (±30%)

Part 1: Dose escalation: CD19 CAR-NK cells-800×10⁶ CD19-CAR+ viable CB-NK cells (±25%)

Part 1: Dose expansion: r/r LBCL: CD19 CAR-NK cells-200×10⁶/800×10⁶ Viable NK Cells

Part 1: Dose expansion: r/r iNHL: CD19 CAR-NK cells-200×10⁶/800×10⁶ Viable NK Cells

Based on the data in Part 1, a single CD19 CAR-NK cells dose level are selected by the sponsor and investigators as the recommended phase 2 dose (RP2D). Once RP2D is determined, participants will be enrolled in Part 2 of the study in the following cohorts:
Cohort 1: CD19 CAR-NK cells (LBCL)
Cohort 2: CD19 CAR-NK cells (iNHL)

The overall time to participate in this study is 5 years. Participants make multiple visits to the clinic and will enroll in a separate, long-term, follow-up study for continued safety assessments for up to 15 years after CD19 CAR-NK cell administration.

The outcome measures for evaluating the effect of CD19 CAR-NK cell administration include monitoring for Adverse Events (AEs), clinically significant changes in Laboratory Parameters (e.g., include hematology, clinical chemistry, serum immunoglobulin and urinalysis tests), clinically significant changes in vital signs (e.g., body temperature (oral or tympanic measurement), sitting blood pressure (after the participant has rested for at least 5 minutes), and pulse rate (bpm)) and/or Overall Response Rate (ORR) per Independent Review Committee (IRC). ORR is defined as the percentage of participants with complete response (CR) or partial response (PR) as best response to treatment, determined by the IRC per the Lugano 2014 criteria after CD19 CAR-NK cells administration.

Secondary Outcome Measures include:
ORR per the Lugano 2014 criteria after CD19 CAR-NK cells administration
Complete Response (CR) per Investigator (CR is determined per Lugano 2014 criteria as percentage of participants with target nodes/nodal masses must regress to ≤1.5 cm in the longest transverse diameter of all lesions and no extralymphatic sites of disease)
Complete Response (CR) Per IRC (per Lugano criteria as percentage of participants with target nodes/nodal masses must regress to ≤1.5 cm in the longest transverse diameter of all lesions and no extralymphatic sites of disease).
Duration of Response (DOR) per Investigator (DOR is defined as the time from the date of first documented objective response to the date of first documented disease progression, determined by investigator per Lugano 2014 criteria classification or death, whichever comes first, for participants who experience an objective response).
Duration of Response (DOR) per IRC (DOR is defined as the time from the date of first documented objective response to the date of first documented disease progression, determined by the IRC Lugano 2014 criteria classification or death, whichever comes first, for participants who experience an objective response)
Progression-free Survival (PFS) per Investigator (PFS is defined as time from enrollment date to the date of disease progression, determined by the investigator per Lugano 2014 criteria classification or death from any cause, whichever comes first)
Progression-free Survival (PFS) per IRC (PFS is defined as time from enrollment date to the date of disease progression, determined by the IRC per Lugano 2014 criteria classification or death from any cause, whichever comes first.)
Overall Survival (OS) (OS is defined as time from enrollment to the date of death from any cause.)
Cmax—Maximum Observed Blood Concentration of CD19 CAR-NK cells
Tmax—Time of First Occurrence of Cmax of CD19 CAR-NK cells
Tlast—Time of Last Measurable Concentration Above the Lower Limit of Quantitation of CD19 CAR-NK cells.
AUClast—Area Under the Concentration-time Curve From Time 0 to Time of the Last Quantifiable Concentration of CD19 CAR-NK cells
Concentration of Interleukin (IL)-15 and Other Soluble Immune Factors in Plasma Over Time
Concentration of IL-15 and soluble immune factors (e.g., Interferon (IFN)-gamma (γ), IL-1 beta (B), IL-2, IL-4, IL-6, IL-8, IL-10, IL-12p70, IL-13, Tumor necrosis factor (TNF) alpha (α), Granulocyte-macrophage colony-stimulating factor (GM-CSF)) in plasma over time will be reported.
Percentage of Participants with B-cell Aplasia Before and After CD19 CAR-NK cells Administration
Percentage of Participants with Detectable Anti-human Leukocyte Antigen (HLA) and Anti-chimeric Antigen Receptor (CAR) Antibodies Before (Prevalence) and After (Incidence) CD19 CAR-NK cells Administration Over Time
Percentage of Participants with Positive Replication Competent Retrovirus (RCR) Test Results Before (Prevalence) and After (Incidence) CD19 CAR-NK cells Administration Over Time

Patients with relapsed/refractory B-cell lymphoma who were previously treated with CD19-targeted therapy, receiving the chimeric antigen receptor (CAR)-NK cell therapy targeting CD19 were evaluated as described above in a single-arm, open label, multicenter, global, phase 2 clinical trial, were evaluated efficacy of the CAR-NK cells (cord blood derived NK cells exogenously transduced anti-CD19 CAR comprising sequences represented by SEQ ID Nos: 1-5, IL-15 represented by SEQ ID No: 6, and iCasp9 represented by SEQ ID No: 7). For the frozen formulation, each cell sample was formulated in 40% PLASMA-LYTE A, 50% CS10, 10% HSA and 30 mM trehalose (e.g., 37.7% v/v PLASMA-LYTE A+50% v/v CS10+9.4% v/v HSA+2.8% v/v trehalose).

An exemplary responding patient demonstrated a partial response within one month following treatment with 800×10⁶ CD19 CAR+ viable NK cells. The patient had iNHL and previously received 8 prior lines of therapy including two CD19-targeting CAR-T cells (axicabtagene ciloleucel and tisagenlecleucel). The partial response converted into complete response after 3 months of receiving the CAR-NK cell infusion (genetically modified umbilical cord blood-derived natural killer cells (NK Cells) transduced with CAR19-CD28-zeta-2A-IL15 and inducible caspase-9).

Efficacy of the CAR-NK cells, on patients with negative CD19 expression was analyzed by immunohistochemistry (IHC). A screening biopsy of an exemplary responding patient showed CD19-negative LBCL in immunohistochemistry. The patient received 800×10⁶ CD19 CAR+ viable NK cells and achieved a partial response within one month following the treatment.

### Example 2. In Vitro Cytotoxicity Assessment on CD19 KO Cancer Cells

This example demonstrates in vitro cytotoxicity analysis to evaluate CD19-independent activity of the CAR-NK cells, that are cord blood derived NK cells exogenously transduced anti-CD19 CAR comprising sequences represented by SEQ ID Nos: 1-5, IL-15 represented by SEQ ID No: 6, and iCasp9 represented by SEQ ID No: 7, on CD19 KO cancer cells. In vitro cytotoxicity of the CAR-NK cells was evaluated against two CD19 negative (CD19-; Raji CD19 knockout [KO], NALM6 CD19 KO) target cell lines. Target cancer cell lines used in these studies expressed NKG2D stress ligands. The two cell types were assessed using flow cytometry-based dead cell evaluation methodologies (lots of the CAR-NK cells tested included: donor A, which had a CAR % of 75.28% and donor B, which had a CAR % of 80.08%).

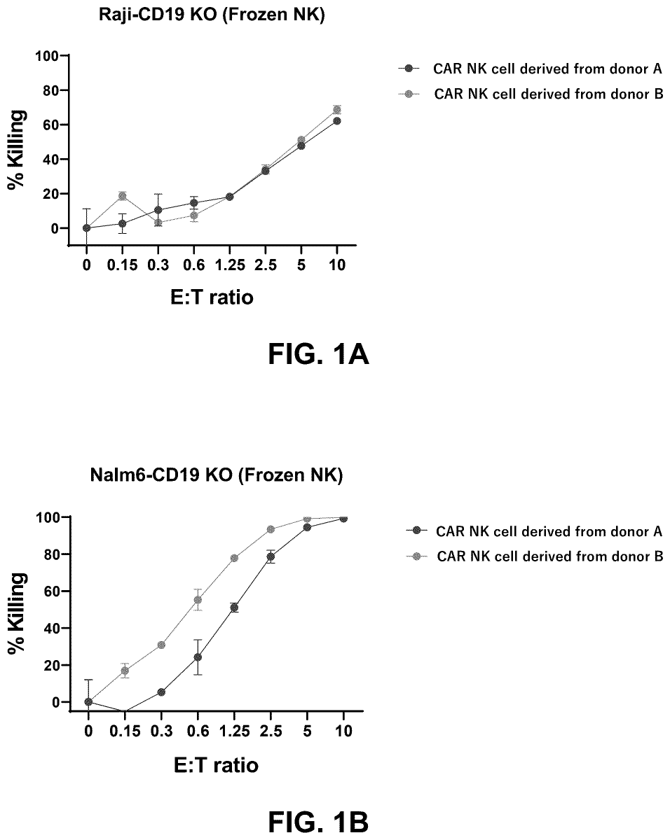

The cytolytic activity of the CAR-NK cells, generated from two independent donor cord blood units (Donor A and Donor B), were evaluated on Raji CD19 KO (FIG. 1A) and NALM6 CD19 KO (FIG. 1B) tumor cells after 20 hours of co-culture. Raji CD19 KO and NALM6 CD19 KO cell lines were freshly thawed, resuspended, labelled with CellTrace Violet and plated at 30,000 cells/100 μL/well. Frozen CAR-NK cells derived from donor A and frozen the CAR-NK cells derived from donor B (for the frozen formulation, each cell sample was formulated in the formulation consisting of 40% PLASMA-LYTE A, 50% CS10, 10% HSA and 30 mM trehalose) were thawed, resuspended in growth medium and viable cells counted. The CAR-NK cells were co-incubated with respective target cells at seven different effector (e.g., the CAR-NK cells) to target cell ratios (10:1, 5:1, 2.5:1, 1.25:1, 0.63:1, 0.3:1, and 0.16:1), respectively. The target cells were pre-labeled with a fluorescent dye (CellTrace Violet) to allow their discrimination from the effector cells. After a 20-hour incubation period at 37° C., cells were washed with staining buffer, resuspended in phosphate buffered saline (PBS) pre-mixed with fixable Viability dye (eFluor 780), incubated in the dark at 40° C. for 30 minutes. The cells were then washed twice before determining the killing activity of the different effector cell treatments by flow cytometry. Target cells alone were used as the baseline of viable cells with no killing. Data represent mean of duplicate wells. Killing or specific lysis of target cells was calculated as follows:
Killing or specific lysis of target cells (%)=100−([number of CellTrace violet positive target cells in wells co-cultured with effector cells/average number of CellTrace violet positive cells in target cell alone controls]*100).

The CAR-NK cells from the two donors demonstrated killing of both cell types in an E:T cell ratio-dependent manner. The CAR-NK cells demonstrated killing of both CD19 negative (Raji-CD19 KO and NALM6-CD19 KO) cancer cells tested, consistent with innate NK receptor engagement of stress ligands expressed on cancer cells.

### Example 3: Combination Therapy of CAR NK Cells to an Individual

This example describes exemplary administration of chemotherapy agents and intravenous CD19 CAR NK cells where the NK cells are derived from core blood to a cancer patient, such as a patient who has Relapsed or Refractory (r/r) B-cell Non-Hodgkin Lymphoma (NHL). Patients with a history of anti-CD19 therapy (e.g., CD19-targeted chimeric antigen receptor (CAR) T cells or monoclonal antibodies) are included. Patients are administered CD19 CAR-NK cells in combination with chemotherapy agents (e.g., fludarabine and cyclophosphamide as per standard of care).

Experimental: Part 1: Dose Escalation: CD19 CAR-NK cells—200×10\hat{ }6 CD19-CAR+ viable CB-NK cells. Participants receive lymphodepleting chemotherapy per day intravenously followed by 200×10\hat{ }6 anti-CD19 chimeric antigen receptor (CD19-CAR+) viable natural killer (NK) cells, single-dose, intravenously, once on Day 0.

Experimental: Part 1: Dose Escalation: CD19 CAR-NK cells—800×10\hat{ }6 CD19-CAR+ viable CB-NK cells Participants receive lymphodepleting chemotherapy per day intravenously followed by CD19 CAR-NK cells—800×10\hat{ }6 CD19-CAR+ viable CB-NK cells, single-dose, intravenously, once on Day 0.

Experimental: Part 1: Dose Expansion: LBCL: CD19 CAR-NK cells—200×10\hat{ }6/800×10\hat{ }6 CD19-CAR+ viable CB-NK cells. Participants with r/r Large B-cell Lymphoma (LBCL) receive lymphodepleting chemotherapy per day intravenously followed by CD19 CAR-NK cells—200×10\hat{ }6/800×10\hat{ }6 CD19-CAR+ viable CB-NK cells, single-dose, intravenously, once on Day 0 to determine RP2D.

Experimental: Part 1: Dose Expansion: iNHL: CD19 CAR-NK cells-200×10\hat{ }6/800×10\hat{ }6 CD19-CAR+ viable CB-NK cells. Participants with r/r Indolent Non-Hodgkin Lymphoma (iNHL) receive lymphodepleting chemotherapy per day intravenously followed by CD19 CAR-NK cells-200×10\hat{ }6/800×10\hat{ }6 CD19-CAR+ viable CB-NK cells, single-dose, intravenously, once on Day 0 to determine RP2D.

Experimental: Part 2: Cohort 1-LBCL. Participants with LBCL are enrolled in this cohort to receive lymphodepleting chemotherapy per day intravenously followed by CD19 CAR-NK cells at RP2D, intravenously, once on Day 0.

Experimental: Part 2: Cohort 2-iNHL

Participants with iNHL are enrolled in this cohort to receive lymphodepleting chemotherapy per day intravenously followed by CD19 CAR-NK cells at RP2D, intravenously, once on Day 0.

Outcomes are assessed as described in Example 1.

### EQUIVALENTS AND SCOPE

Those skilled in the art will recognize or be able to ascertain using no more than routine experimentation, many equivalents to the specific embodiments of the invention described herein. The scope of the present invention is not intended to be limited to the above Description, but rather is as set forth in the following claims:

1. A method of treating cancer in an individual, comprising a step of administering a therapeutically effective amount of CD19-CAR+ viable cord blood natural killer (CB-NK) cells to the individual, wherein the individual has previously received a CD19 targeted CAR T therapy.

2. The method of claim 1, wherein the patient has previously received at least 2 lines of standard chemoimmunotherapy or targeted therapy.

3. The method of claim 1 or 2, wherein the individual is administered lymphodepleting chemotherapy intravenously followed by administration of CD19-CAR+ viable CB-NK cells.

4. The method of any one of the preceding claims, wherein the individual previously received anti-CD19 CAR-T therapy more than three months prior to administration of the CD19-CAR+ viable CB-NK cells.

5. The method of any one of the preceding claims, wherein the cancer is a solid tumor or is not a solid tumor.

6. The method of any one of the preceding claims, wherein the cancer is of the lung, brain, breast, blood, skin, pancreas, liver, colon, head and neck, kidney, thyroid, stomach, spleen, gallbladder, bone, ovary, testes, endometrium, prostate, rectum, anus, cervix, or is hematological.

7. The method of any one of the preceding claims, wherein the cancer is relapsed or refractory B-cell Non-Hodgkin Lymphoma.

8. A method of treating cancer in an individual, comprising a step of administering a therapeutically effective amount of CD19-CAR+ viable cord blood natural killer (CB-NK) cells to the individual, wherein the cancer is CD19 negative.

9. The method of claim 8, wherein the cancer is a solid tumor or is not a solid tumor.

10. The method of claim 8, wherein the cancer is large B-cell lymphoma.

11. The method of any one of the preceding claims, wherein CD19-CAR+ viable CB-NK cells are administered at a dose of at least 200×10⁶.

12. The method of any one of the preceding claims, wherein the individual is a human.

13. The method of any one of the preceding claims, wherein the individual is administered one or more additional cancer therapies.

14. The method of claim 13, wherein the additional cancer therapy is surgery, radiation, chemotherapy, hormone therapy, immunotherapy, or a combination thereof.

15. The method of any one of the preceding claims, further comprising a step of diagnosing cancer in the individual.

16. The method of any one of the preceding claims, further comprising a step of generating the CD19-CAR+ viable CB-NK cells.

17. The method of any one of the preceding claims, wherein the cells are autologous with respect to the individual.

18. The method of any one of the preceding claims, wherein the cells are allogeneic with respect to the individual.

19. The method of any one of the preceding claims, wherein the population of cells are administered to the individual intracranially, by injection, intravenously, intraarterially, intraperitoneally, intratracheally, intratumorally, intramuscularly, endoscopically, intralesionally, intracranially, percutaneously, subcutaneously, regionally, by perfusion, in a tumor microenvironment, or a combination thereof.

20. The method of any one of the preceding claims, wherein the CD19-CAR+ Viable (NK) cells comprise one or more exogenously provided interleukins (IL).

21. The method of claim 20, wherein the IL is selected from the group consisting of IL-12, IL-15, IL-21, IL-2, IL-18, IL-7, the p35 and p40 subunits of IL-12 artificially linked together with a linker, and a combination thereof.

22. The method of claim 20, wherein the IL is IL-15.

23. The method of any one of claims 20-22, wherein said IL is secreted, tethered, or membrane bound in the cell.

24. The method of any one of claims 20-23, wherein exogenously provided IL is expressed from a vector in the cells and/or wherein the NK cells are cultured in the presence of one or more IL.

25. The method of any one of the preceding claims, wherein the NK cell comprises a suicide gene.

26. The method of claim 25, wherein the suicide gene is an iCaspase9 suicide gene.
